(19) United States
(12) Patent Application Publication
(10) Pub. No.: US 2021/0101869 A1
(43) Pub. Date: Apr. 8, 2021

(54) IONIC COMPOUNDS COMPRISING 1,3-DIALKYL-4,5,6,7-TETRAHYDRO-1H-BENZO[D]IMIDAZOL-3-IUM CATIONS FOR USE IN COATINGS AND ADHESIVES

(71) Applicant: NITTO DENKO CORPORATION, Osaka (JP)

(72) Inventors: Stanislaw Rachwal, Oceanside, CA (US); Yufen Hu, San Diego, CA (US); Hongxi Zhang, Temecula, CA (US); Peng Wang, San Diego, CA (US)

(21) Appl. No.: 16/500,053

(22) PCT Filed: Apr. 10, 2018
(86) PCT No.: PCT/US2018/026847

§ 371 Date: Oct. 1, 2019

Related U.S. Application Data
(60) Provisional application No. 62/484,211, filed on Apr. 11, 2017.

(51) Int. Cl. C07D 235/04; B32B 15/082; B32B 7/12

(52) U.S. Cl. CPC C07D 235/04; B32B 15/082; B32B 7/12; B32B 2255/06; B32B 2307/202; B32B 2307/752; B32B 2457/00; B32B 2255/24; B32B 2255/26

(57) ABSTRACT

The present disclosure relates to ionic compositions having hydrophobic characteristics and/or reduced corrosiveness upon application to metallic substrates. An ionic composition, which may be used as an adhesive material for selectively adhering two electroconducting surface together is also described herein, wherein the application of electromotive force to the electroconducting materials can reduce the adhesion of the adhesive material. Some embodiments provide a hydrophobic ionic debonding compound include a benzimidazolium cation and a sulfonylimide anion.

### CROSS-REFERENCES TO RELATED APPLICATION

This application claims the benefit of U.S. Provisional Patent Application No. 62/484,211 filed Apr. 11, 2017, the content of which is incorporated herein by reference in its entirety.

### FIELD

This disclosure relates to ionic liquids for use as coatings and adhesives that can be separated or removed from a surface to which they are applied by applying electromotive force to the compound or material.

### BACKGROUND

Certain electro-debonding compositions are known which may be used as an adhesive coating, such as the composition comprising commercial 1-ethyl-3-methyl-imidazolium bis(fluorosulfonyl)imide, sulfonylimide, functionalized ionic liquids, or imidazolium analogues used in power storage devices. However, the composition comprising 1-ethyl-3-methyl-imidazolium and/or bis(fluorosulfonyl)imide as coating material can be relatively corrosive to an aluminum surface.

Additionally most ionic liquids are strongly hydrophilic and hygroscopic. They dissolve in water well and absorb water readily from the ambient atmosphere. In applications where dry environment is required, such ionic liquids are not suitable to use.

Thus, there is a need for a new hydrophobic ionic compound or composition that can be debonded from a surface without corrosiveness to the metallic substrates once coated.

### SUMMARY

This disclosure relates to an ionic compound comprising a 4,5,6,7-tetrahydro-benzoimidazolium cation, such as a compound according to Formula 2:

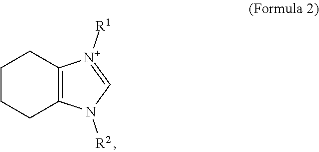

wherein R¹ and R² are independently optionally substituted C₂₋₇ alkyl or optionally substituted —(C₁₋₃ alkyl)—O—(C₁₋₃ alkyl).

Some embodiments include a composition comprising an ionic compound containing a 4,5,6,7-tetrahydro-benzoimidazolium cation. Some embodiments include a composition comprising an ionic compound containing a 4,5,6,7-tetrahydro-benzoimidazolium cation, further comprising an anion according to Formula 2:

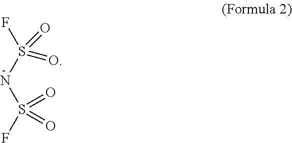

Some embodiments include an ionic composition having hydrophobic characteristic and having reduced corrosion upon application to metallic substrates. Some embodiments include the ionic composition described herein that can be used as a coating or an adhesive deposited on a substrate, which can be disbanded or removed from a surface to which it is applied without damage to that surface upon an application of electromotive force. Such an ionic composition can further comprising an acrylic polymer to form an electrically debondable adhesive composite, which can be in a form of layer or sheet upon coating or depositing on a substrate, such as a conductive substrate.

Some embodiments include a device, comprising: a first electroconducting substrate; a second electroconducting substrate; and a composition or an ionic compound described herein, positioned between the first electroconducting substrate and the second electroconducting substrates, and binding the first electroconducting substrate to the second electroconducting substrate. In some embodiments, the composition or ionic compound used in the device has the property that an application of 10 volts electric potential difference between the first electroconducting substrate and the second electroconducting substrates reduces the adhesion of the selectively adhesive material.

Some embodiments include a method comprising applying a composition or an ionic compound described herein between a first electroconducting substrate and a second electroconducting to adhere the first electroconducting substrate to the second electroconducting substrate.

### BRIEF DESCRIPTION OF THE DRAWINGS

FIG. 1 is a schematic diagram of a device incorporating an embodiment of the use of an ionic compound or composition described herein.

FIG. 2 is a schematic diagram of a device incorporating an embodiment of the use of an ionic compound or composition described herein.

FIG. 3 is a schematic diagram of a device used in testing the adhesion properties of the ionic compounds described herein.

FIG. 4 is a plot of peeling strength density vs. time of an ionic compound or composition described herein tested in the device shown in FIG. 3.

### DETAILED DESCRIPTION

As used herein, when a compound or chemical structural is referred to as being “optionally substituted”, it includes a feature that has no substituents (i.e. unsubstituted), or a feature that is “substituted,” meaning that the feature has one or more substituents. The term “substituent” is broad, and includes a moiety that occupies a position normally occupied by one or more hydrogen atoms attached to a parent compound or structural feature. Any suitable substituent may be used, including substituents having a molecular weight of 15-50 g/mol, 15-100 g/m, or 15-200 g/mol, and substituents composed of atoms such as C, H, N, O, S, F, Cl, Br, and/or I. In some embodiments, substituent groups are independently optionally substituted alkyl, alkenyl, alkoxy (e.g. —OCH₃, —OC₂H₅, —OC₂H₅, —OC₄H₉, etc.), alkylsulfones (e.g. —SCH₃, —SC₂H₅, —SC₃H₇, —SC₄H₉, etc.), —NR′R″, —OH, —SH, —CN, —NO₂, or a halogen, wherein R′ and R″ are independently H or optionally substituted alkyl. As used herein, the term “4,5,6,7-tetrahydro-benzoimidazolium cation” refers to the ring system having the following structure:

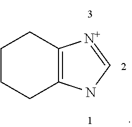

As used herein, the terms “bis(sulfonyl)imide and/or “sulfonyl imide” refers to a heteroatom moiety, for example, having the following structure:

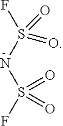

As used herein, the term “hydrophilic and/or hydrophilicity” refers to the preference of an ionic liquid (IL) to stay in water over an organic solvent. One useful test for determining relative hydrophobicity or hydrophilicity is to measure the partition ratio (or IL/DPM) of the test compound relative to an internal reference standard hydrophobic compound such as diphenylmethane (DPM) in an organic solvent. The partition ratio (or IL/DPM) is obtained by measuring the preference of the ionic liquid to stay in ethyl acetate over water relative to diphenyl methane as an internal reference standard. Typically, compounds having a partition ratio (IL/DPM) of less than about 0.5 would be considered hydrophilic.

As used herein, the term “hydrophobic and/or hydrophobicity” refers to the feature of an ionic liquid having preference of staying in an organic phase over water. Typically, compounds having a partition ratio (or IL/DPM) of greater than about 0.5 would be hydrophobic.

Some embodiments include an ionic liquid compound comprising an optionally substituted 4,5,6,7-tetrahydro-benzoimidazolium cation according to Formula 1:

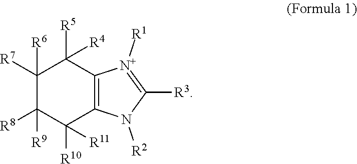

R¹ and R² are independently optionally substituted C₂₋₈ alkyl, or optionally substituted —(C₁₋₆ alkyl)—O—(C₁₋₆ alkyl); R³ is a substituent such as H or C₁₋₃ alkyl; and R⁴, R⁵, R⁶, R⁷, R⁸, R⁹, R¹⁰, or R¹¹ are independently any substituents such as the substituents as described below.

With respect of Formula 1, in some embodiments, R³, R⁴, R⁵, R⁶, R⁷, R⁸, R⁹, R¹⁰ and R¹¹ are all H, and the ionic liquid compound comprises a 4,5,6,7-tetrahydro-benzoimidazolium cation according to Formula 2:

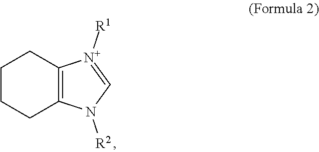

wherein, R¹ and R² are independently optionally substituted C₂₋₇ alkyl, or optionally substituted C₁₋₇ alkoxyalkyl.

With respect to any relevant structural representation, such as Formula 1 or Formula 2, in some embodiments, R¹ is: optionally substituted C₂₋₇ alkyl, such as optionally substituted C₂ alkyl (e.g. ethyl), C₃ alkyl (e.g. propyl), C₄ alkyl (e.g. butyl), C₅ alkyl (e.g. pentyl), C₆ alkyl (e.g. hexyl), or C₇ alkyl (e.g. heptyl). In some embodiments, R¹ is optionally substituted C₁₋₇ alkoxyalkyl, such as —(C₁₋₃ alkyl)—O—(C₁₋₃ alkyl), e.g. —(C₂ alkyl)—O—(C₁₋₃ alkyl) or —(C₂ alkyl)—O—(C₁₋₃ alkyl). In some embodiments, R¹ is ethyl. In some embodiments, R¹ is isobutyl. In some embodiments, R¹ is neopentyl. In some embodiments, R¹ is n-hexyl. In some embodiments, R¹ is 2-ethoxyethyl.

With respect to any relevant structural representation, such as Formula 1 or Formula 2, in some embodiments, R² is: optionally substituted C₂₋₇ alkyl, such as optionally substituted C₂ alkyl (e.g. ethyl), C₃ alkyl (e.g. propyl), C₄ alkyl (e.g. butyl), C₅ alkyl (e.g. pentyl), C₆ alkyl (e.g. hexyl), or C₇ alkyl (e.g. heptyl). In some embodiments, R² is optionally substituted C₁₋₇ alkoxyalkyl, such as —(C₁₋₃ alkyl)—O—(C₁₋₃ alkyl), e.g. —(C₂ alkyl)—O—(C₁₋₃ alkyl) or —(C₂ alkyl)—O—(C₁₋₃ alkyl). In some embodiments, R² is ethyl. In some embodiments, R² is isobutyl. In some embodiments, R² is neopentyl. In some embodiments, R² is n-hexyl. In some embodiments, R² is 2-ethoxyethyl.

With respect to Formula 1 or Formula 2, in some embodiments, both R¹ and R² are ethyl. In some embodiments, both R¹ and R² are n-hexyl. In some embodiments, both R¹ and R² are isobutyl. In some embodiments, R¹ is neopentyl, and R² is n-hexyl. In some embodiments, R¹ is 2-ethoxyethyl, and R² is n-hexyl.

In some embodiments, IV, R², or both R¹ and R² of Formula 1 or 2 can be hydrophilic functional group. In some embodiments, at least one of R¹ and R² can be hydrophilic functional group. In some embodiments, the hydrophilic functional group can comprise oxygen. In some embodiments, the hydrophilic functional group can be ether, hydroxyl, alkoxyl, or ester group.

In some embodiments, R¹, R², or both R¹ and R² of Formula 1 or 2 can be hydrophobic functional group. In some embodiments, at least one of R¹ and R² can be a hydrophobic functional group. In some embodiments, the hydrophobic functional group can comprise an optionally substituted alkyl group. In some embodiments, the optionally substituted alkyl group can comprise methyl, ethyl, propyl, butyl, pentyl, hexyl, or hepatyl group.

Some embodiments include an ionic compound of Formula 1 or 2, comprising a 4,5,6,7-tetrahydro-benzoimidazolium cation that is symmetrical. In some embodiments, the ionic cation described herein is asymmetrical.

In some embodiments, R¹ and R² of Formula 1 or 2 are independently:

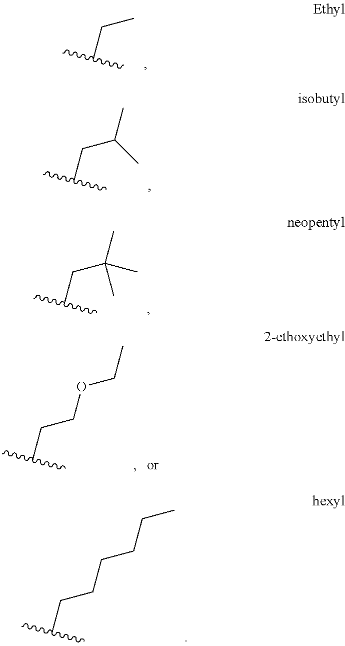

In some embodiments, 4,5,6,7-tetrahydro-benzoimidazolium cation of Formula 1 or 2 is:

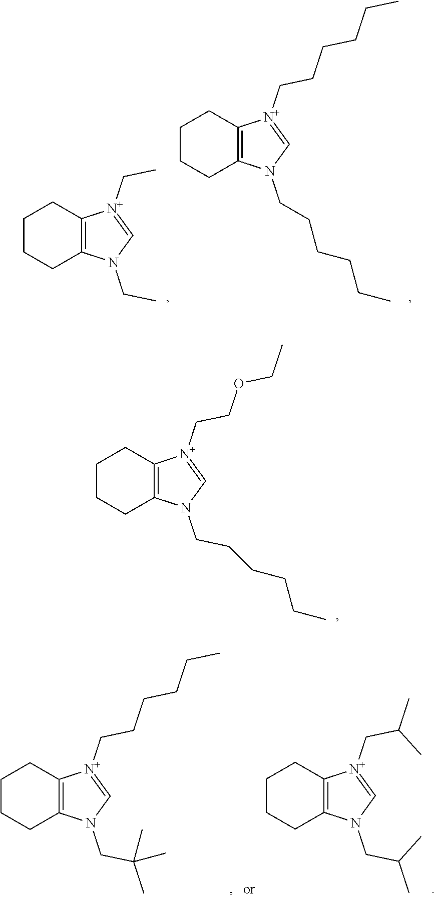 [SMILES: CCn1c[n+](CC)c2c1CCCC2 ; CCCCCCn1c[n+](CCCCCC)c2c1CCCC2 ; CCCCCCn1c[n+](CCOCC)c2c1CCCC2 ; CCCCCC[n+]1cn(CC(C)(C)C)c2c1CCCC2 ; CC(C)Cn1c[n+](CC(C)C)c2c1CCCC2]

With respect to any relevant structural representation, such as Formula 1, in some embodiments, R³ is H, CH₃, C₂H₅, C₃H₇. In some embodiments, R³ is H.

With respect to any relevant structural representation, such as Formula 1, in some embodiments, R⁴ is H, CH₃, C₂H₅, C₃H₇, CH₃O (e.g. —OCH₃ or —CH₂OH), C₂H₅O, or C₃H₇O. In some embodiments, R⁴ is H.

With respect to any relevant structural representation, such as Formula 1, in some embodiments, R⁵ is H.

With respect to any relevant structural representation, such as Formula 1, in some embodiments, R⁶ is H, CH₃, C₂H₅, C₃H₇, CH₃O (e.g. —OCH₃ or —CH₂OH), C₂H₅O, or C₃H₇O. In some embodiments, R⁶ is H.

With respect to any relevant structural representation, such as Formula 1, in some embodiments, R⁷ is H.

With respect to any relevant structural representation, such as Formula 1, in some embodiments, R⁸ is H, CH₃, C₂H₅, C₃H₇, CH₃O (e.g. —OCH₃ or —CH₂OH), C₂H₅O, or C₃H₇O. In some embodiments, R⁸ is H.

With respect to any relevant structural representation, such as Formula 1, in some embodiments, R⁹ is H.

With respect to any relevant structural representation, such as Formula 1, in some embodiments, R¹⁰ is H, CH₃, C₂H₅, C₃H₇, CH₃O (e.g. —OCH₃ or —CH₂OH), C₂H₅O, or C₃H₇O. In some embodiments, R¹⁰ is H.

With respect to any relevant structural representation, such as Formula 1, in some embodiments, R¹¹ is H.

In some embodiments, the ionic composition can further comprise a bis(sulfonyl)imide anion, such as an anion with a structure shown in Formula 3:

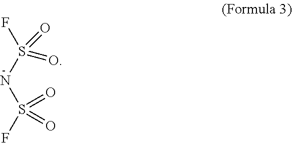

Some embodiments include a selectively adhesive material, comprising the ionic compositions described herein. The selectively adhesive material comprising the ionic composition described above can bind a first electro-conducting surface and a second electro-conducting surface together, wherein an application of electromotive force to the electro-conductive surfaces reduces the adhesion of the adhesive material. Some of the selectively adhering adhesive compositions comprise a 4,5,6,7-tetrahydro-benzoimidazolium cation described above. In some embodiments, the selectively adhesive composition, containing a 4,5,6,7-tetrahydro-benzoimidazolium cation, can further comprising a sulfonylimide anion, such as bis(fluorosulfonyl)imide anion described above.

Some of the selectively adhering adhesive described herein can further comprise a polymer to form a composite adhesive. The glass transition temperature of the polymer can be below about 0° C. In some embodiments, the polymer may be a polymer described in JP 2015-204998 and/or in JP 2015-204996. In some embodiments, the polymer can be an acrylic polymer. The acrylic polymer can contain a monomer unit derived from a C₁₋₁₄ alkyl (or alkoxy) group containing alkyl (meth)acrylate ester, such as butyl (meth)acrylate. In some embodiments, the monomer unit comprises a carboxyl group

Some embodiments include a device comprising any of the aforementioned ionic compounds or compositions. A suitable example of such a device can be as that described in JP 2015-204996 and/or in JP20204997.

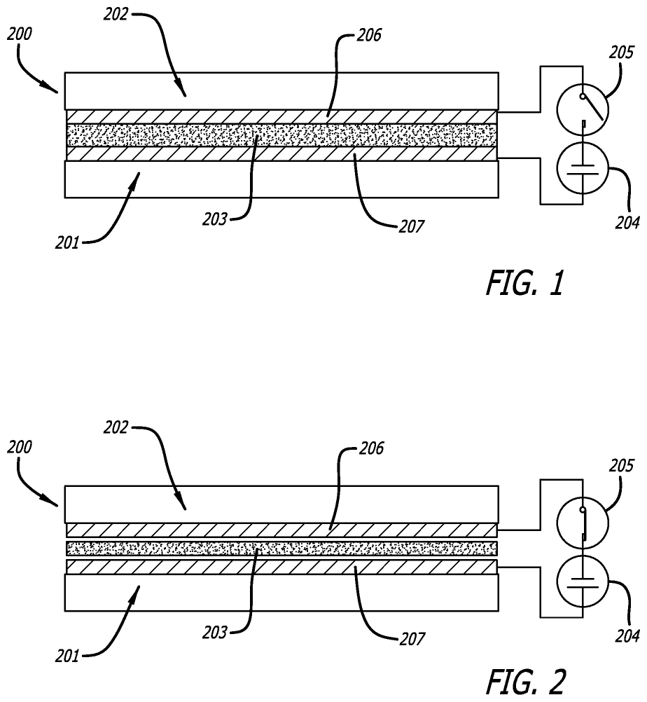

FIGS. 1 and 2 show some possible embodiments such as 200 of a composition described herein binding and connecting a first electro-conducting surface and a second electro-conducting surface together. As shown in FIGS. 1 and 2, adhesive 203, incorporating the ionic compounds or compositions described herein, can be an adhesive layer or a coating disposed between first electro-conducting substrate 206 and second electro-conducting substrate 207. First electro-conducting substrate 206 and second electro-conducting substrate 207 can disposed upon two non-metal (non-electro-conducting) substrates or layers, 201 and 202 respectively. The first and second electro-conducting substrates can be in electrical communication with a power supply 204, e.g., a direct current power supply, to complete a closeable electrical circuit with an intervening switch 205. When the switch 205 is closed, either or both of the two non-metal adherents 201 and 202 can separate the adhesive 203 from the either or both of the first and second electro-conducting substrates (separating metal and coating/adhesive at interfaces).

Any suitable electromotive force may be used to reduce the adhesion of the composition described herein so that the composition may be separated from one or two surfaces to which the composition is adhered. In some embodiments, the DC voltage can be about 3-100 V, about 3-5 V, about 5-10 V, about 10-20 V, about 20-30 V, about 30-50 V, about 50-70 V, about 60-80 V, about 80-100 V, about 10 V, or any DC voltage in a range bounded by any of these values.

Typically, when the composition is intended to be separated from one or more surfaces, the electromotive force that reduces the adhesive force between the composition and the surface can be smaller than the actual electromotive force applied to the composition. In some embodiments, a DC voltage of about 3-90 V, about 3-5 V, about 5-10 V, about 10-20 V, about 20-30 V, about 30-50 V, about 50-70 V, about 60-80 V, about 80-90 V, about 10 V, or any DC voltage in a range bounded by any of these values, may reduce the adhesive force between the composition and the surface.

In some embodiments, the electro-conductive substrate can comprise an electro-conducting carbonaceous material. In some embodiments, the electro-conductive substrate can comprise an electroconducting metal. A conductive metal layer can comprise any electro-conducting metal, for example aluminum. The conductive metal layer may comprise a conventional material, such as a metal, mixed metal, alloy, metal oxide, and/or composite metal, or a conductive polymer. Examples of suitable metals include the Group 1 metals, the metals in Groups 4, 5, 6, and the Group 8-10 transition metals in the Periodical Table. Examples of suitable metals include stainless steel, Al, Ag, Mg, Ca, Cu, Mg/Ag, LiF/Al, CsF, and/or CsF/Al, and/or alloys thereof. In some embodiments, the electro-conducting layers can have a thickness of about 1 nm to about 1000 μm. In some embodiments, the electro-conducting layer has a thickness of about 20 nm to about 200 μm, about 20-200 nm, about 20-50 nm, about 50-100 nm, about 100-200 nm, about 200-500 nm, about 500-1000 nm, about 1-100 μm, about 100-200 μm, about 200-500 μm, about 500-100 μm, about 40-60 nm, or about 50 nm, or any thickness in a range bound by any of these values.

In some embodiments, the selectively adherent adhesive comprising the ionic liquid compound described herein can have a reduced corrosive effect on the electro-conducting layer coated with such an adhesive. In some embodiments, the selectively adherent adhesive comprising an ionic compound described herein can be used to coat on an electro-conductive layer to reduce the acidity of the environment immediately adjacent to the electro-conductive layer. Suitable methods to assess the corrosive effect of the adhesive coated or deposited on the electro-conducting materials can be the methods described in ASTM G69-12 (Standard Test Method for Measurement of Corrosion Potentials of Aluminum Alloys).

In some embodiments, the selectively adherent adhesive composition comprising an ionic compound described herein can be used to coat or deposit on a substrate. The adhesive composition can be in a form of a layer or a sheet. The thickness of the layer or the sheet can be about 5 μm to about 100 μm, 5 μm to about 150 μm, about 5 μm to about 50 μm, about 5 μm to about 10 μm; about 10 μm to about 50 μm, about 50 μm to about 100 μm, or about 100 μm to about 150 μm, or any thickness in a range bounded by any of these values.

In some embodiments, the selectively adherent adhesive composition comprising an ionic compound described herein, can further comprise an acrylic polymer, which can be used to coat or deposit on a substrate to form a composite composition. This composite composition can be in a form of a layer or a sheet. The thickness of the layer or the sheet comprising the composite composition can be about 50 μm to about 100 μm, 50 μm to about 150 μm, about 50 μm to about 200 μm, about 50 μm to about 100 μm, or about 100 μm to about 150 μm, or any thickness in a range bounded by any of these values.

In some embodiments, the selectively adherent adhesive comprising the ionic compounds described herein can be chemically stable on an electrically conductive electrode or electro-conducting materials. In some embodiments, the electrically conductive electrode can comprise an aluminum, stainless steel, and/or combinations or mixtures thereof. In some embodiments, chemical stability is defined as lack of (or minimal presence of) undesired reactions between a metal anode and the selectively adherent adhesive. Undesired reactions may include, for example, corrosive degradation of the metal electrode, dissolution of the metal in the selectively adherent adhesive, and/or pitting of the metal electrode. In some embodiments, the aforementioned ionic composition deposited on, or in contact with the electrically conductive metal electrode may result in a reduced or absence of corrosive degradation mentioned above. In some embodiments, direct contact of the neat ionic compound upon the electrically conductive metal electrode may show any corrosive degradation mentioned above for a period of at least (or greater than) about 15 minutes, at least about 30 minutes, at least about 1 hour, at least about 3 hours, at least about 5 hours, at least about 7 hours, at least about 24 hours, at least about 50 hours, at least about 70 hours, at least about 100 hours, at least about 125 hours, at least about 200 hours, at least about 300 hours, or at least about 450 hours, or any time period in a range bounded by any of these values. In some embodiments, direct contact of the neat ionic composition upon the electrically conductive electrode may minimize and/or prevent corrosive degradation for a period of time as described above. In some embodiments, direct contact of the neat ionic composition upon the electrically conductive electrode may minimize and/or prevent corrosive degradation for a period of time as described above even under a high temperature of 85° C., and high relative humidity of 85%. In some embodiments, no corrosive degradation is observed, as it lacks total penetration of an electro-conducting 50 nm-thick sheet of aluminum foil with the above described time frames, and/or environmental conditions.

In some embodiments, the selectively adhesive compositions described herein can be formulated to minimize corrosion of the above-described electro-conducting substrates coated with such compositions, under conditions of high humidity and high temperature for prolonged time period. In particular, the adhesive composition can be capable of maintaining two such electro-conducting substrates in fixed relation to each other during and after being exposed to 85° C. and 85% relative humidity for a period of time described above.

In some embodiments, the hydrophobicity of the ionic materials described herein can be determined by measuring the partition of the ionic materials between aqueous and organic phases. A suitable system utilizing water/ethyl acetate can be used to measure hydrophilic-hydrophobic behavior of ionic liquids. Diphenylmethane (DPM) can be used as an internal standard as a hydrophobic compound (assuming 100% hydrophobic). Thus, a solution of ionic liquid (0.5 mmol) and diphenylmethane (0.5 mmol) in ethyl acetate (100 mL) can be shaken with water (100 mL). After phase separation, ethyl acetate in the organic phase can be removed by evaporation under reduced pressure to leave a mixture of ionic liquid and DPM. The molar ratio of these two chemicals can be measured by Nuclear Magnetic Resonance (NMR) spectroscopy. The value obtained by this test, or the ratio: (moles ionic liquid/moles diphenylmethane), is referred to herein as “partition ratio” or “IL/DPM.” If IL/DPM is more than 0.5, suggesting that the ionic liquid prefers to stay in ethyl acetate phase over water phase, then the ionic liquid is considered hydrophobic. Likewise, If an ionic liquid prefers to stay in water phase over ethyl acetate phase (IL/DPM<0.5), the ionic liquid is considered hydrophilic. In some embodiments, IL/DPM can be about 0.77 indicating that the ionic liquid is moderate hydrophobic. In some embodiment, IL/DPM can be as high as 1.0, which is considered strongly hydrophobic. Some ionic compounds are hydrophobic having IL/DPM greater than 0.5 and up to 1.0.

In some embodiments, the hydrophobicity of the materials can be determined by measuring the Water Contact Angle (WCA). Higher water contact angles are indicative of increased hydrophobicity. In other words, the greater the WCA, the less spread out a drop of water is on a surface. Increased water contact angles are indicative of less surface wetting and thus increased hydrophobicity. However, the hydrophobicity of a solution can also be measured by dropping it on an oil-compatible substrate, such as a polymer (e.g., PMMA). In this case, the relationship is reversed, and lower Solution Contact Angle (SCA) can be indicative of higher hydrophobicity. In some embodiments, the compounds described herein can have a SCA of less than about 35°, about 30-35 degree, about 25-30 degree, about 20-25 degree, about 15-20 degree, about 10-15 degree, about 10-20 degree, about 30°, about 25°, about 20°, about 18°, or about 16°.

In some embodiments, the hydrophobicity of the ionic materials can be observed as being insoluble in water, but soluble in a water-miscible organic solvent.

The following embodiments are specifically contemplated:

### EMBODIMENTS

Embodiment 1. An ionic compound comprising a cation according to the formula:

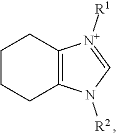

wherein R¹ and R² are independently C₂₋₇ alkyl or —(C₁₋₃ alkyl)—O—(C₁₋₃ alkyl).

Embodiment 2. The ionic compound of embodiment 1, wherein R¹ is C₂ alkyl, C₃ alkyl, C₄ alkyl, C₅ alkyl, C₆ alkyl, or C₇ alkyl.

Embodiment 3. The ionic compound of embodiment 1, wherein R¹ is ethyl.

Embodiment 4. The ionic compound of embodiment 1, wherein R¹ is isobutyl.

Embodiment 5. The ionic compound of embodiment 1, wherein R¹ is neopentyl.

Embodiment 6. The ionic compound of embodiment 1, wherein R¹ is n-hexyl.

Embodiment 7. The ionic compound of embodiment 1, wherein R¹ is —(C₂ alkyl)—O—(C₁₋₃ alkyl) or —(C₂ alkyl)—O—(C₁₋₃ alkyl).

Embodiment 8. The ionic compound of embodiment 1, wherein R¹ is 2-ethoxyethyl.

Embodiment 9. The ionic compound of embodiment 1, 2, 3, 4, 5, 6, 7, or 8, wherein R² is C₂ alkyl, C₃ alkyl, C₄ alkyl, C₅ alkyl, C₆ alkyl, or C₇ alkyl.

Embodiment 10. The ionic compound of embodiment 1, 2, 3, 4, 5, 6, 7, or 8, wherein R² is ethyl.

Embodiment 11. The ionic compound of embodiment 1, 2, 3, 4, 5, 6, 7, or 8, wherein R² is isobutyl.

Embodiment 12. The ionic compound of embodiment 1, 2, 3, 4, 5, 6, 7, or 8, wherein R² is neopentyl.

Embodiment 13. The ionic compound of embodiment 1, 2, 3, 4, 5, 6, 7, or 8, wherein R² is n-hexyl.

Embodiment 14. The ionic compound of embodiment 1, 2, 3, 4, 5, 6, 7, or 8, wherein R² is 2-ethoxyethyl.

Embodiment 15. The ionic compound of embodiment 1, comprising:

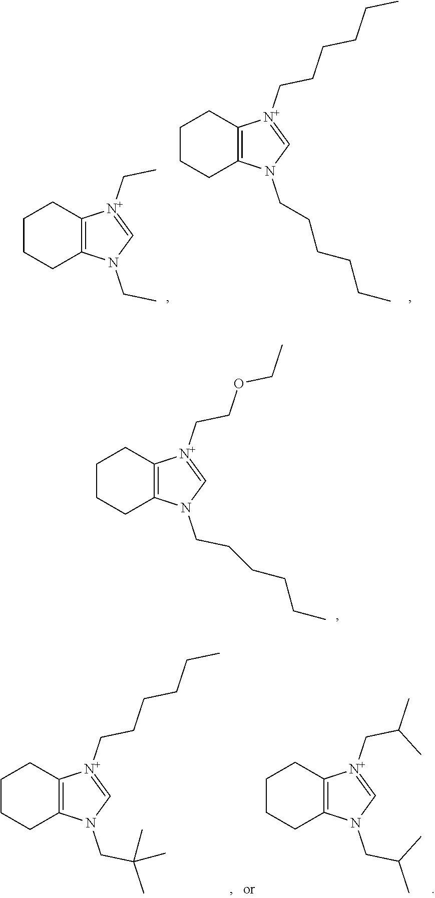 [SMILES: CCn1c[n+](CC)c2c1CCCC2 ; CCCCCCn1c[n+](CCCCCC)c2c1CCCC2 ; CCCCCCn1c[n+](CCOCC)c2c1CCCC2 ; CCCCCC[n+]1cn(CC(C)(C)C)c2c1CCCC2 ; CC(C)Cn1c[n+](CC(C)C)c2c1CCCC2]

Embodiment 16. The ionic compound of embodiment 1, 2, 3, 4, 5, 6, 7, 8, 9, 10, 11, 12, 13, 14, or 15, having a partition ratio (IL/DPM) that is about 0.5 to about 1.0.

Embodiment 17. The ionic compound of embodiment 1, 2, 3, 4, 5, 6, 7, 8, 9, 10, 11, 12, 13, 14, or 15, having a partition ratio (IL/DPM) that is about 0.7 to about 0.9.

Embodiment 18. The ionic compound of embodiment 1, 2, 3, 4, 5, 6, 7, 8, 9, 10, 11, 12, 13, 14, or 15, having a partition ratio (IL/DPM) that is about 0.9 to about 1.0.

Embodiment 19. The ionic compound of embodiment 1, 2, 3, 4, 5, 6, 7, 8, 9, 10, 11, 12, 13, 14, 15, 16, 17, 18, or 19, further comprising an anion according to the following formula:

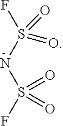

Embodiment 20. A composition, comprising an ionic compound of embodiment 1, 2, 3, 4, 5, 6, 7, 8, 9, 10, 11, 12, 13, 14, 15, 16, 17, 18, or 19.

Embodiment 21. The composition of embodiment 20, which is in the form of a coating or an adhesive.

Embodiment 22. The composition of embodiment 21, having an adhesive strength that reduces upon application of a 10-volt electric potential difference to the composition.

Embodiment 23. The composition of embodiment m 20, 21, or 22, which is less corrosive than 1-ethyl-3-methyl-imidazolium bis(fluorosulfonyl)imide, as determined by the amount of contact time required to visually observe corrosion after directly applying the composition or 1-ethyl-3-methyl-imidazolium bis(fluorosulfonyl)imide to an aluminum foil.

Embodiment 24. The composition of embodiment 20, 21, 22, or 23, having the property that no corrosion on coated aluminum foil is visually observed for at least about 70 hours of contact time at the interface between the composition and the surface of an aluminum foil at a temperature of 85° C. and a humidity of 85%.

Embodiment 25. The composition of embodiment 20, 21, 22, 23, or 24, further comprising an acrylic polymer to form a composite layer or a sheet.

Embodiment 26. The composition of embodiment 21, 22, 23, 24, or 25, which is in the form of a layer or a sheet on a substrate.

Embodiment 27. The composition of embodiment 26, wherein the layer or sheet has a thickness of about 5 μm to about 150 μm.

Embodiment 28. The composition of embodiment 25, wherein the composite layer or sheet has a thickness of about 150 μm.

Embodiment 29. The composition of embodiment 26, 27, or 28, wherein the substrate is a conductive substrate.

Embodiment 30. The composition of embodiment 29, wherein the conductive substrate is a metal film, or a metalized plastic film, or a combination thereof.

Embodiment 31. The composition of embodiment 30, wherein the metal film is an aluminum foil, and the metalized plastic film is PET.

Embodiment 32. A device, comprising:

a first electroconducting substrate;

a second electroconducting substrate; and

a composition of embodiment 20, 21, 22, 23, 24, 25, 26, 27, 28, 29, 30, or 31, positioned between the first electroconducting substrate and the second electroconducting substrates, and binding the first electroconducting substrate to the second electroconducting substrate;

wherein the composition has the property that an application of 10 volts electric potential difference between the first electroconducting substrate and the second electroconducting substrate reduces the adhesion of the composition.

Embodiment 33. A method comprising applying the composition of embodiment 20, 21, 22, 23, 24, 25, 26, 27, 28, 29, 30, or 31 between a first electroconducting substrate and a second electroconducting to adhere the first electroconducting substrate to the second electroconducting substrate.

Embodiment 34. The method of embodiment 33, further comprising applying an electrical current between the first electroconducting substrate and the second electroconducting substrate to separate the first electroconducting substrate or the second electroconducting substrate or both electroconducting substrates from the composition.

### EXAMPLES

Described herein are ionic compositions and elements that can reduce the deterioration and/or corrosion of the conductive metal layers described herein. These benefits are further shown by the following examples, which are intended to be illustrative of the embodiments of the disclosure, but are not intended to limit the scope or underlying principles in any way.

The abbreviation for some of the compounds or solvents used in the following examples are described below:

KOH: potassium hydroxide

DCM: dichloromethane

DMSO: dimethyl sulfoxide

KFSI: potassium fluorosilicate

THF: tetrahydrofuran

### Example 1: 1,3-Diethyl-4,5,6,7-tetrahydro-1H-benzo[d]imidazol-3-ium bis(fluorosulfonyl)amide (S1)

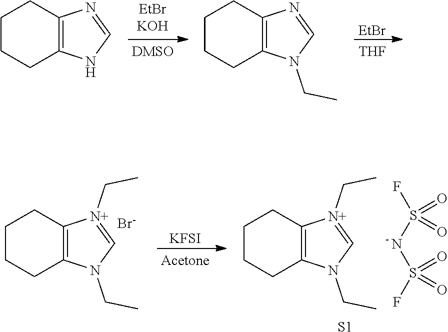

A mixture of 4,5,6,7-tetrahydro-1H-benzo[d]imidazole (3.60 g, 28.5 mmol), 90% KOH (2.49 g, 40 mmol) and DMSO (60 mL) was stirred under argon at room temperature for 16 h. Ethyl bromide (2.6 mL, 35 mmol) was added dropwise while the reaction mixture was cooled externally to keep its temperature in the range of 15-20° C. Stirring at room temperature was then continued for 6 h. The reaction mixture was poured into ice/water (500 mL), treated with 5 N NaOH (100 mL) and extracted with DCM (2×200 mL). The extract was washed with water (200 mL), dried over Na₂CO₃, and the solvent was removed under reduced pressure to give 1-ethyl-4,5,6,7-tetrahydro-1H-benzo[d]imidazole (2.17 g, 49% yield).

A solution of 1-ethyl-4,5,6,7-tetrahydro-1H-benzo[d]imidazole (2.00 g, 13.3 mmol) and ethyl bromide (1.00 mL) in dry THF (20 mL) was stirred under argon and heated at 60° C. for 30 h. Two layers were formed. Upon standing at room temperature overnight, the bottom layer solidified. The solid was separated and recrystallized from acetone with cooling in dry ice to give 1,3-diethyl-4,5,6,7-tetrahydro-1H-benzo[d]imidazol-3-ium bromide (1.95 g, 56% yield).

A mixture of 1,3-diethyl-4,5,6,7-tetrahydro-1H-benzo[d]imidazol-3-ium bromide (1.87 g, 7.21 mmol), potassium salt of bis(fluorosulfonyl)imide (KFSI) (1.58 g, 7.21 mmol) and acetone (20 mL) was stirred under argon and heated at 50° C. for 3 h. After cooling to room temperature, the solid was filtered off, and the solvent was removed under reduced pressure. A solution of the residue in anhydrous THF (50 mL) was kept at room temperature overnight, then filtered, and concentrated under reduced pressure to give 1,3-diethyl-4,5,6,7-tetrahydro-1H-benzo[d]imidazol-3-ium bis(fluorosulfonyl)amide, S1 (2.50 g, 96% yield). ¹H NMR (DMSO-d₆): δ 9.10 (s, 1H), 4.10 (q, J=7.5 Hz, 4H), 2.62 (bs, 4H), 1.79 (bs, 4H), 1.39 (t, J=7.5 Hz, 6H).

### Example 2: 1,3-Dihexyl-4,5,6,7-tetrahydro-1H-benzo[d]imidazol-3-ium bis(fluorosulfonyl)amide (S2)

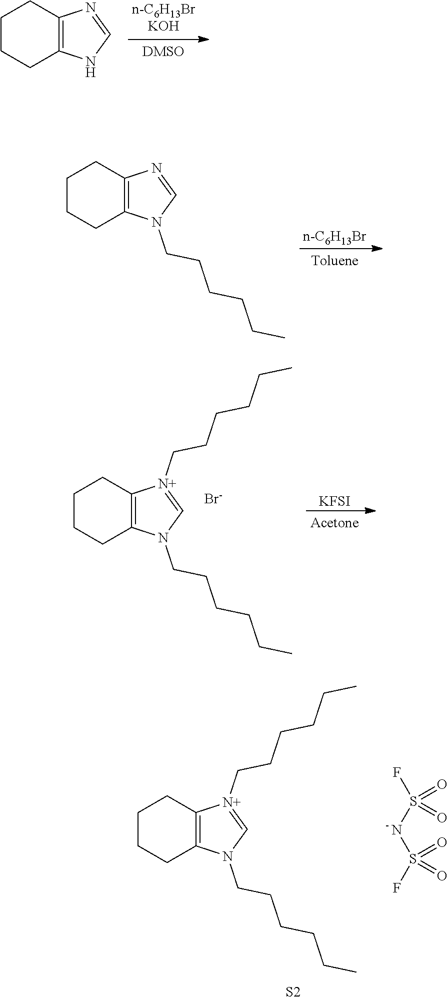

A mixture of 4,5,6,7-tetrahydro-1H-benzo[d]imidazole (5.00 g, 40.9 mmol), 90% KOH (3.74 g, 60 mmol) and DMSO (70 mL) was stirred under argon and heated at 40° C. for 2 h. After cooling to 10° C., 1-bromohexane (6.3 mL, 45 mmol) was added in one portion, and the resulting mixture was stirred and heated at 40° C. for 4 days. The reaction mixture was poured into ice/water (600 mL), treated with 5 N NaOH (100 mL) and extracted with DCM (2×400 mL). The extract was washed with water (300 mL), dried over Na₂SO₄, and the solvent was removed under reduced pressure to give a residue. Column chromatography of the residue (silica gel, ethyl acetate/methanol, 97:3) afforded pure 1-hexyl-4,5,6,7-tetrahydro-1H-benzo[d]imidazole (6.54 g, 77% yield).

A solution of 1-hexyl-4,5,6,7-tetrahydro-1H-benzo[d]imidazole (3.45 g, 16.7 mmol) and 1-bromohexane (3.5 mL, 25.0 mmol) in dry toluene (50 mL) was stirred under argon and heated at 100° C. for 24 h. The volatiles were removed under reduced pressure. The residue was triturated with ethyl ether (50 mL). The oily bottom phase was separated and dried in a vacuum oven to give 1,3-dihexyl-4,5,6,7-tetrahydro-1H-benzo[d]imidazol-3-ium bromide (5.99 g, 96% yield).

A mixture of 1,3-dihexyl-4,5,6,7-tetrahydro-1H-benzo[d]imidazol-3-ium bromide (2.89 g, 7.78 mmol), KFSI (1.705 g, 7.78 mmol) and acetone (50 mL) was stirred under argon and heated at 50° C. for 2 h. After cooling to room temperature, the solid was filtered off, and the solvent was removed under reduced pressure to give a residue. A solution of the residue in anhydrous THF (100 mL) was kept at room temperature overnight, then filtered, and concentrated under reduced pressure. A solution of the crude product in ethyl acetate (100 mL) was washed with water (2×100 mL), dried over Na₂SO₄, and the solvent was removed under reduced pressure to give 1,3-dihexyl-4,5,6,7-tetrahydro-1H-benzo[d]imidazol-3-ium bis(fluorosulfonyl)amide, S2 (3.50 g, 95% yield). ¹H NMR (DMSO-d₆): δ 9.02 (s, 1H), 4.05 (t, J=7.5 Hz, 4H), 2.61 (bs, 4H), 1.80 (bs, 4H), 1.74 (quintet, J=6.5 Hz, 4H), 1.28 (bs, 12H), 0.87 (t, J=7.0 Hz, 6H) ppm.

### Example 3: 3-(2-Ethoxyethyl)-1-hexyl-4,5,6,7-tetrahydro-1H-benzo[d]imidazol-3-ium bis(fluorosulfonyl)amide (S3)

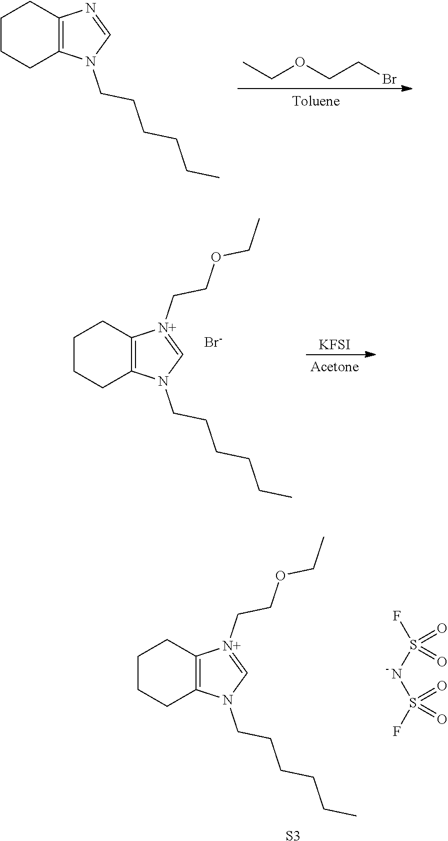

A solution of 1-hexyl-4,5,6,7-tetrahydro-1H-benzo[d]imidazole (3.10 g, 15.0 mmol) and 2-bromoethyl ethyl ether (2.8 mL, 25.0 mmol)) in dry toluene (50 mL) was stirred under argon and heated at 100° C. for 24 h. The volatiles were removed under reduced pressure. The residue was triturated with ethyl ether (50 mL). The oily bottom phase was separated and dried in a vacuum oven to give 3-(2-ethoxyethyl)-1-hexyl-4,5,6,7-tetrahydro-1H-benzo[d]imidazol-3-ium bromide (5.18 g, 96% yield).

A mixture of 3-(2-ethoxyethyl)-1-hexyl-4,5,6,7-tetra hydro-1H-benzo[d]imidazol-3-ium bromide (5.16 g, 14.4 mmol), KFSI (3.15 g, 14.4 mmol) and acetone (50 mL) was stirred under argon and heated at 50° C. for 2 h. After cooling to room temperature, the solid was filtered off, and the solvent was removed under reduced pressure to give a residue. A solution of the residue in anhydrous THF (100 mL) was kept at room temperature overnight, then filtered and concentrated under reduced pressure to provide a crude product. A solution of the crude product in ethyl ether/ethyl acetate (50 mL/50 mL) was washed with water (50 mL), dried over Na₂SO₄, and the solvent was removed under reduced pressure to give 3-(2-ethoxyethyl)-1-hexyl 4,5,6,7-tetrahydro-1H-benzo[d]imidazol-3-ium bis(fluorosulfonyl)amide, S3 (5.37 g, 81% yield). ¹H NMR (DMSO-d₆): δ 8.96 (s, 1H), 4.25 (t, J=5.0 Hz, 2H), 4.08 (t, J=7.5 Hz, 2H), 3.67 (t, J=5.0 Hz, 2H), 3.44 (q, J=7.0 Hz, 2H), 2.62 (bs, 4H), 1.79 (bs, 4H), 1.72 (quintet, J=6.5 Hz, 2H), 1.28 (bs, 6H), 1.07 (t, J=7.0 Hz, 3H), 0.87 (t, J=7.0 Hz, 3H) ppm.

### Example 4: 1-Hexyl-3-neopentyl-4,5,6,7-tetrahydro-1H-benzo[d]imidazol-3-ium bis(fluorosulfonyl)amide (S4)

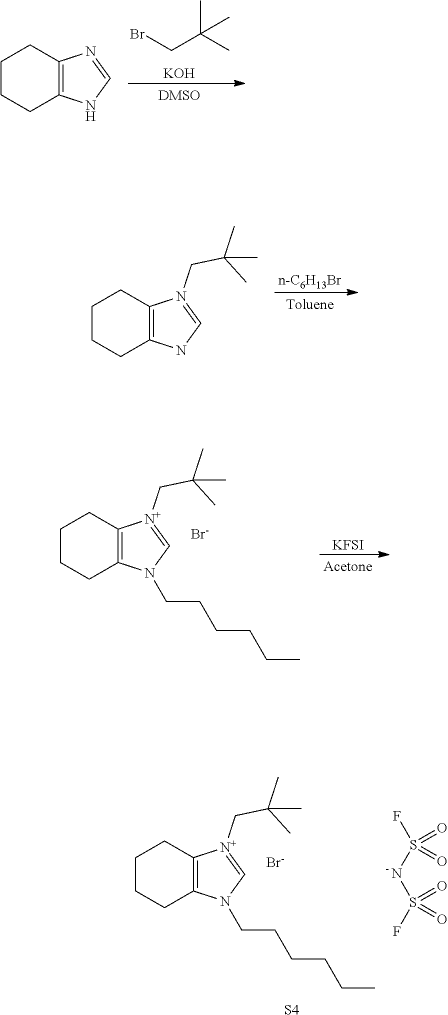

A mixture of 4,5,6,7-tetrahydro-1H-benzo[d]imidazole (5.00 g, 40.9 mmol), 90% KOH (3.74 g, 60 mmol) and DMSO (70 mL) was stirred under argon and heated at 40° C. for 2 h. After cooling to 10° C., neopentyl bromide (5.5 mL, 44 mmol) was added in one portion, and the resulting mixture was stirred and heated at 40° C. for 4 days. The reaction mixture was poured into ice/water (800 mL), treated with 5 N NaOH (200 mL), and extracted with DCM (2×400 mL). The extract was washed with water (200 mL), dried over Na₂SO₄, and the solvent was removed under reduced pressure to give a residue. Column chromatography of the residue (silica gel, ethyl acetate/methanol, 95:5) afforded pure 1-neopentyl-4,5,6,7-tetrahydro-1H-benzo[d]imidazole (4.72 g, 60% yield).

A solution of 1-neopentyl-4,5,6,7-tetrahydro-1H-benzo[d]imidazole (4.60 g, 23.9 mmol) and 1-bromohexane (8.0 mL, 57.0 mmol) in dry toluene (75 mL) was stirred under argon and heated at 100° C. for 24 h. The volatiles were removed under reduced pressure. The residue was triturated with ethyl ether (50 mL) and separated by decantation. To remove its dark color, the product was dissolved in methanol (100 mL), treated with activated carbon and stirred overnight. The carbon was filtered off using a pad of Celite, and the solvent was removed under reduced pressure to give pure 1-hexyl-3-neopentyl-4,5,6,7-tetrahydro-1H-benzo[d]imidazol-3-ium bromide (5.00 g, 58% yield).

A mixture of 1-hexyl-3-neopentyl-4,5,6,7-tetrahydro-1H-benzo[d]imidazol-3-ium bromide (5.00 g, 14.0 mmol), KFSI (3.07 g, 14.0 mmol) and acetone (60 mL) was stirred under argon and heated at 60° C. for 2 h. After cooling to room temperature, the solid was filtered off, and the solvent was removed under reduced pressure. A solution of the residue in anhydrous THF (100 mL) was kept at room temperature overnight, then filtered and concentrated under reduced pressure to give 1-hexyl-3-neopentyl-4,5,6,7-tetrahydro-1H-benzo[d]imidazol-3-ium bis(fluorosulfonyl)amide, S4 (6.20 g, 97% yield). ¹H NMR (DMSO-d₆): δ 8.99 (s, 1H), 4.10 (t, J=7.0 Hz, 2H), 3.90 (s, 2H), 2.61 (m, 4H), 1.78 (m, 4H), 1.74 (m, 2H), 1.26 (bs, 6H), 0.95 (s, 9H), 0.86 (t, J=7.0 Hz, 3H) ppm.

### Example 5: 1,3-Diisobutyl-4,5,6,7-tetrahydro-1H-benzo[d]imidazol-3-ium bis(fluorosulfonyl)amide (55)

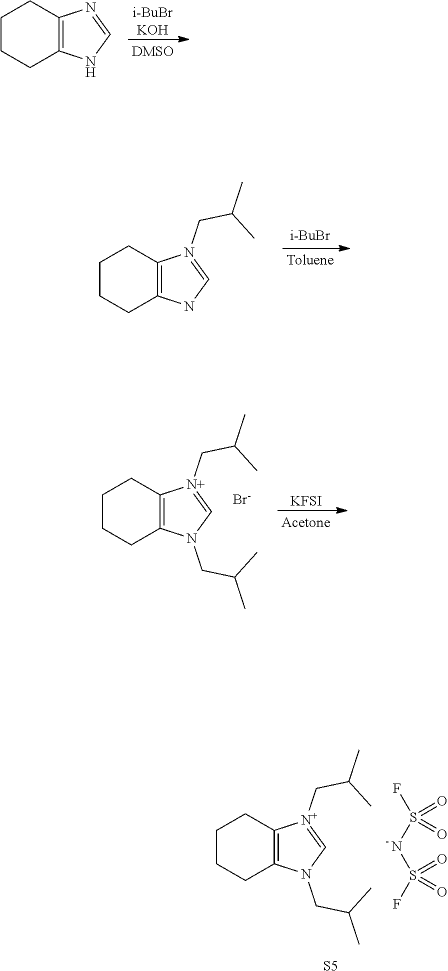

A mixture of 4,5,6,7-tetrahydro-1H-benzo[d]imidazole (6.00 g, 49.1 mmol), 90% KOH (4.35 g, 70 mmol) and DMSO (80 mL) was stirred under argon and heated at 50° C. for 1 h. After cooling to 10° C., isobutyl bromide (5.7 mL, 52 mmol) was added portion-wise within 1 h while keeping temperature in the range of 10-15° C., and the resulting mixture was then stirred at room temperature for 40 hours. The reaction mixture was poured into ice/water (400 mL), treated with 5 N NaOH (100 mL) and extracted with DCM (2×200 mL). The extract was washed with water (200 mL), dried over Na₂SO₄, and the solvent was removed under reduced pressure to give a residue. Column chromatography of the residue (silica gel, ethyl acetate/methanol, 95:5) afforded pure 1-isobutyl-4,5,6,7-tetrahydro-1H-benzo[d]imidazole (5.62 g, 64% yield).

A mixture of 1-isobutyl-4,5,6,7-tetrahydro-1H-benzo[d]imidazole (5.50 g, 30.8 mmol), isobutyl bromide (5.4 mL, 50 mmol) and toluene (75 mL) was stirred under argon and heated at 100° C. for 16 h. TLC indicated that only about a half of the starting material reacted. Temperature was increased to 120° C., and the reaction was continued for additional 20 h. The solvent was removed under reduced pressure. The residue was washed thoroughly with ethyl ether and dried in a vacuum oven at 80° C. to give 1,3-diisobutyl-4,5,6,7-tetrahydro-1H-benzo[d]imidazol-3-ium bromide (5.50 g, 57% yield).

A mixture of 1,3-diisobutyl-4,5,6,7-tetrahydro-1H-benzo[d]imidazol-3-ium bromide (4.80 g, 15.2 mmol), KFSI (3.29 g, 15.0 mmol) and acetone (50 mL) was stirred under argon and heated at 50° C. for 2 h. After cooling to room temperature, the solid was filtered off, and the solvent was removed under reduced pressure. A solution of the residue in ethyl acetate (150 mL) was washed with water (100 mL) adjusted pH of the aqueous layer to 8.0 with saturated sodium bicarbonate, dried over anhydrous sodium sulfate, and concentrated under reduced pressure to give a residue. The residue was triturated with a mixture of ethyl ether/hexanes (1:1, 100 mL) to give crystalline 1,3-diisobutyl-4,5,6,7-tetrahydro-1H-benzo[d]imidazol-3-ium bis(fluorosulfonyl)amide, S5 (4.79 g, 76% yield). ¹H NMR (DMSO-d₆): δ 9.02 (s, 1H), 3.92 (d, J=7.5 Hz, 4H), 2.61 (bs, 4H), 2.02 (m, 2H), 1.80 (bs, 4H), 0.89 (d, J=6.5 Hz, 12H) ppm.

### Example 6: Measuring Hydrophobicity of Ionic Liquids

A system containing water/ethyl acetate was designed to measure hydrophilic-hydrophobic behavior of ionic liquids. Diphenylmethane (DPM) is used as an internal standard. Thus, a solution of ionic liquid (0.5 mmol) and diphenylmethane (0.5 mmol) in ethyl acetate (100 mL) was shaken with water (100 mL). After phase separation, ethyl acetate in the organic layer was removed by evaporation under reduced pressure to leave a mixture of ionic liquid and DPM. The molar ratio of these two chemicals was then determined by NMR analysis. Assuming DPM represents to be 100% hydrophobic, the molar ratio of ionic liquid to DPM indicates how hydrophobic an ionic liquid is. For the purposes of this disclosure, the value obtained by this test will referred to as the “partition ratio,” or “IL/DPM.” If an ionic liquid (IL) prefers to stay in water over organic layer (such as ethyl acetate) (IL/DPM <0.5), we call it hydrophilic. If an ionic liquid prefers to stay in organic layer (such as ethyl acetate) over aqueous layer, (IL/DPM >0.5), we call it hydrophobic. The data for two ionic liquids (S1 and S2) and a reference compound CE-1 are given below.

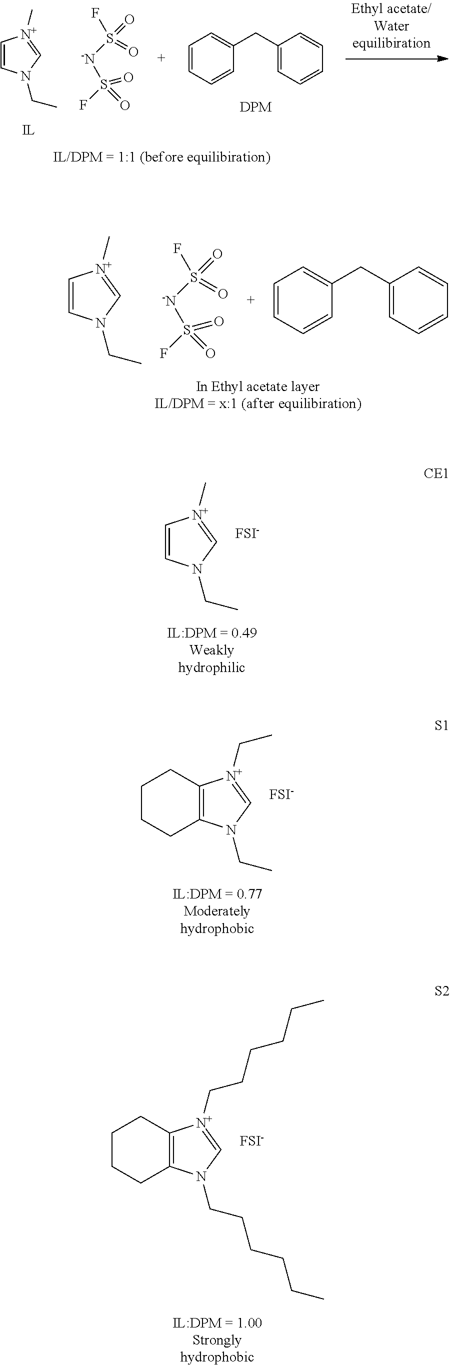

### Example 7: Preparation of Polymer Solution

n-Butyl acrylate (BA) (95 mass parts), acrylic acid (AA) (5 mass parts) and ethyl acetate (125 mass parts) were introduced into a flask (containing a stirring bar) attached to a condenser that was equipped with a nitrogen gas inlet. The mixture was stirred at room temperature while introducing the nitrogen gas for about 1 hour to remove oxygen from the reaction system. Azobisisobutyronitrile (AIBN) (0.2 mass parts) was added, which increased the temperature of the resulting mixture to about 63±2° C., and the resulting mixture was stirred for about 5-6 hours for polymerization. After stopping heating, an acrylic polymer-containing solution was obtained with a concentration of about 30 wt %. The apparent molecular weight of the polymer (P1) was determined to be about 800,000, with a Tg (glass transition temperature) of about −50° C.

### Example 8: Preparation of Adhesive Sheet

First, an electrically debondable adhesive composition was obtained by mixing the polymer solution described above with 0.01 gram of an epoxy crosslinking agent, such as N,N,N′,N′-tetraglycidyl-m-xylenediamine, per 100 gram of polymer solution, and one of the ionic liquid compounds (1.5 wt %) described above. The prepared compositions were then coated/deposited upon a surface treated PET separator (release liner) [MRF38, made by Mitsubishi Chemical Corp., Japan], forming an adhesive composite layer at a thickness of about 150 μm (microns). The coated film was then heat dried at 130° C. for about 3 minutes. A second PET separator (release liner) was then aligned over the exposed adhesive coating to obtain a layered sheet (PET separator/adhesive coating/PET separator) which was then aged/dried at 50° C. for about 20-24 hours and then stored under ambient conditions until needed.

### Example 9. Adhesive Ionic Composition Corrosive Test

Just prior to the application of the adhesive sheet to the nano-AI coated layer, the aforementioned release liner was removed. The adhesive sheet, as previously described above was applied to the metallic surface of the aluminum film (50 nm-thick aluminum coated PET film [Toray Advanced Film, Tokyo, Japan]).

The prepared film was placed in a Temperature & Humidity Benchtop chamber, set at 85° C. and 85% Relative Humidity (ESPEC North America, [Hudsonville, Mich., USA], Criterion Temperature & Humidity Benchtop Model BTL-433) and were checked at 70 hours. The interface between the adhesive and the aluminum foil was visually examined for an indication of corrosive degradation of the aluminum foil and/or dissolution of the metal in the selectively adherent adhesive and/or pitting of the aluminum foil. If any of the corrosiveness described herein was not visually observed, the sample was indicated as “No Corrosion”. If any of the corrosiveness described herein was observed and was as extensive as (or close to) the corrosiveness visually observed for CE-1, the sample is indicated as “Excessive Corrosion”. If very little of the corrosiveness described herein was visually observed and was significantly less corrosive than that was visually observed for CE-1, the sample was indicated as “Slight Corrosion”. The results are summarized in Table 1, below.

TABLE 1

| No IL | CE-1 | S1 | S2 | S3 | S4 | S5 |
|---|---|---|---|---|---|---|
| No | Excessive | Excessive | No | Slight | Excessive | No |
| Corro- | Corro- | Corro- | Corro- | Corro- | Corro- | Corro- |
| sion | sion | sion | sion | sion | sion | sion |

### Example 10: Adhesion Testing of Adhesive Compositions

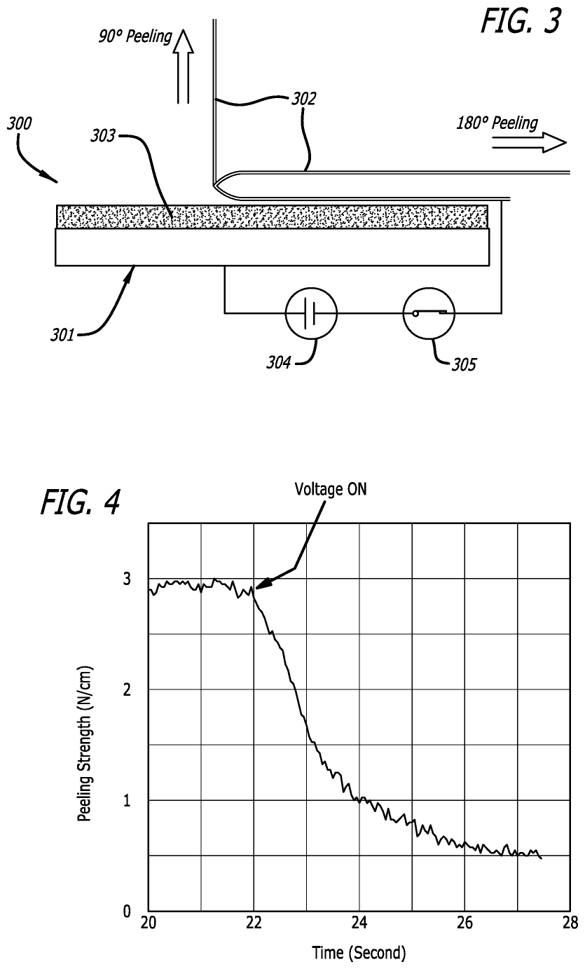

The testing for adhesion was done in the manner as described in JP 2015-228951 and/or JP 2015-204998, and also shown in FIG. 3. As shown in FIG. 3, in the device 300, the adhesive material 303 was coated upon a conductive substrate 301, which is 25 mm wide and 100 mm long, and laminated by the application of rolling pressure, by 2 kg roller and roll press upon another flexible conductive layer 302 (such as aluminum foil and/or metalized plastic film such as PET), which is 10 mm to 25 mm wide and 100 mm longer than conductive substrate 301.

The bonding/de-bonding tester (Mark-10, Copiague, N.Y., USA, model ESM303 motorized tension/compression stand) was equipped with a Mark-10 force gauge (Series 7-1000) and had lower and upper clamps. As shown in FIG. 3, the conductive substrate 301 as described in Example 2 was fixed onto the lower clamp and then electrically connected to the positive pole of a power supply 304 (Protek DC Power Supply 3006B). The top layer 302 was fixed to the upper clamp which is connected with the negative pole of the same power supply which was connected with a power on/off switch 305. The power supply had an output range from 0 to 100 VDC.

In a dynamic test, the moving/peeling speed was set at 300 mm/min., and the voltage was applied a few seconds after the peeling or separation starts. The time and peeling strength readings from the force gauge are recorded by the data acquisition system (Mark-10 MESURgauge Plus). FIG. 4 shows the 180 degree peeling strength evolution over time when a 10 VDC was applied to the adhesive material that is doped with Compound S2 with a concentration of 5 wt. %.

In a static de-bonding test, the sample was fixed on to the tester and connected to the power supply in the same way. The peeling strength of the initial 180-degree peeling was measured at the same peeling speed of 300 mm/min. Then peeling was stopped. A DC voltage (10 VDC for example) was applied for some time (10 second for example). And then the peeling strength was measured at the same peeling speed of 300 mm/min. For the same adhesive sample from compound S2, the initial peeling strength is 3.0 N/cm, and the residual adhesion peeling strength is about 0.2 after applying 10 VDC for 10 second.

Unless otherwise indicated, all numbers expressing quantities of ingredients, properties such as molecular weight, reaction conditions, and so forth used in the specification and embodiments are to be understood as being modified in all instances by the term “about.” Accordingly, unless indicated to the contrary, the numerical parameters set forth in the specification and attached embodiments are approximations that may vary depending upon the desired properties sought to be obtained. At the very least, and not as an attempt to limit the application of the doctrine of equivalents to the scope of the embodiments, each numerical parameter should at least be construed in light of the number of reported significant digits and by applying ordinary rounding techniques.

The terms “a,” “an,” “the” and similar referents used in the context of describing the invention (especially in the context of the following embodiments) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any embodiment. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended embodiments.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the embodiments include all modifications and equivalents of the subject matter recited in the embodiments as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the embodiments. Other modifications that may be employed are within the scope of the embodiments. Thus, by way of example, but not of limitation, alternative embodiments may be utilized in accordance with the teachings herein. Accordingly, the embodiments are not limited to embodiments precisely as shown and described.

1. An ionic compound comprising a cation according to the formula: 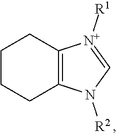
wherein R¹ and R² are independently C₂ alkyl, C₃ alkyl, C₄ alkyl, C₅ alkyl, C₆ alkyl, C₇ alkyl, or —(C₁₋₃ alkyl)—O—(C₁₋₃ alkyl).

2.-34. (canceled)

35. The ionic compound of claim 1, further comprising an anion according to the following formula:

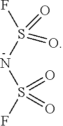

36. The ionic compound of claim 35, wherein R¹ is ethyl.

37. The ionic compound of claim 35, wherein R¹ is isobutyl.

38. The ionic compound of claim 35, wherein R¹ is neopentyl.

39. The ionic compound of claim 35, wherein R¹ is n-hexyl.

40. The ionic compound of claim 35, wherein R¹ is 2-ethoxyethyl.

41. The ionic compound of claim 35, wherein R² is ethyl.

42. The ionic compound of claim 35, wherein R² is isobutyl.

43. The ionic compound of claim 35, wherein R² is neopentyl.

44. The ionic compound of claim 35, wherein R² is n-hexyl.

45. The ionic compound of claim 35, wherein R² is 2-ethoxyethyl.

46. The ionic compound of claim 35, comprising:

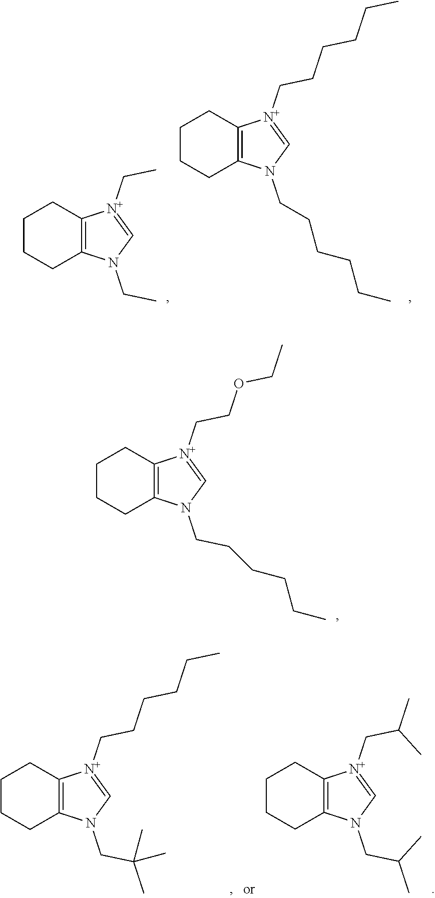 [SMILES: CCn1c[n+](CC)c2c1CCCC2 ; CCCCCCn1c[n+](CCCCCC)c2c1CCCC2 ; CCCCCCn1c[n+](CCOCC)c2c1CCCC2 ; CCCCCC[n+]1cn(CC(C)(C)C)c2c1CCCC2 ; CC(C)Cn1c[n+](CC(C)C)c2c1CCCC2]

47. A composition, comprising an ionic compound of claim 35.

48. The composition of claim 47, further comprising an acrylic polymer to form a composite layer or a sheet.

49. The composition of claim 48, which forms a layer or a sheet on a conductive substrate.

50. The composition of claim 49, wherein the layer or sheet has a thickness of about 5 μm to about 150 μm.

51. The composition of claim 49, wherein the conductive substrate is a metal film, a metalized plastic film, or a combination thereof.

52. The composition of claim 49, which is less corrosive than 1-ethyl-3-methyl-imidazolium bis(fluorosulfonyl)imide, as determined by the amount of contact time required to visually observe corrosion after directly applying the composition or 1-ethyl-3-methyl-imidazolium bis(fluorosulfonyl)imide to an aluminum foil.

53. A device, comprising:
a first electroconducting substrate;
a second electroconducting substrate; and
a composition of claim 47, positioned between the first electroconducting substrate and the second electroconducting substrates, and binding the first electroconducting substrate to the second electroconducting substrate;
wherein the composition has the property that an application of 10 volts electric potential difference between the first electroconducting substrate and the second electroconducting substrate reduces the adhesion of the composition.
